(19) United States
(12) Patent Application Publication
(10) Pub. No.: US 2015/0030688 A1
(43) Pub. Date: Jan. 29, 2015

(54) HONEY AND GROWTH FACTOR ELUTING SCAFFOLD FOR WOUND HEALING AND TISSUE ENGINEERING

(71) Applicant: Saint Louis University, St. Louis, MO (US)

(72) Inventors: Scott Allen Sell, St. Louis, MO (US); Benjamin Alexander Minden-Birkenmaier, St. Louis, MO (US)

(21) Appl. No.: 14/337,925

(22) Filed: Jul. 22, 2014

Related U.S. Application Data
(60) Provisional application No. 61/858,297, filed on Jul. 25, 2013.

(51) Int. Cl. A61K 38/18; A61K 35/64

(52) U.S. Cl. CPC A61K 38/18; A61K 35/644
USPC 424/537; 514/7.6

(57) ABSTRACT

Tissue engineering structures with biologically favorable structural and chemical properties are disclosed. More particularly, the present disclosure is directed to tissue engineered scaffolds having a fiber support and honey. The tissue engineered scaffolds having a fiber support and honey can further include at least one biomolecule. The tissue engineered scaffolds can be used to promote cellular chemotaxis, enhance cell proliferation, enhance extracellular matrix production, increase angiogenesis, and provide antimicrobial activity. The nature of the tissue engineered scaffolds provides a template for cellular infiltration and guide tissue regeneration. The tissue engineered scaffolds can be used in the treatment of dermal wounds (burns, chronic wounds, etc.) or as a tissue engineering scaffold in a wide range of applications.

### CROSS-REFERENCE TO RELATED APPLICATIONS

This application claims priority to U.S. Provisional Patent Application No. 61/858,297, filed on Jul. 25, 2013, which is hereby expressly incorporated by reference in its entirety.

### BACKGROUND

The present disclosure relates generally to tissue engineering structures with biologically favorable structural and chemical properties. More particularly, the present disclosure is directed to tissue engineered scaffolds having a polymeric backbone and incorporating honey and at least one biomolecule. The tissue engineered scaffolds can be used to promote cellular chemotaxis, enhance cell proliferation, enhance extracellular matrix production, increase angiogenesis, and provide antimicrobial activity. The nature of the tissue engineered scaffolds provides a template for cellular infiltration and guide tissue regeneration. The tissue engineered scaffolds can be used in the treatment of dermal wounds (burns, chronic wounds, etc.) or as a tissue engineering scaffold in a wide range of applications.

Honey had been used medicinally for centuries, due to its inherent wound healing capacity. The introduction of penicillin significantly reduced the use of honey in medicinal applications. Recently, with the emergence of antibiotic-resistant bacteria and a better scientific understanding of how honey influences healing, honey (specifically active Leptospermum honey from New Zealand, known as Manuka) has once again become an acceptable product in the treatment of wounds.

The major benefit of Manuka honey lies in its potent antibacterial properties. Honey has a high osmolarity and a high sugar content, the combination of which has been shown to inhibit microbial growth. Manuka honey is also known to have a relatively low pH (3.5-4.5), which, in addition to inhibiting microbial growth, will stimulate the bactericidal actions of macrophages, and in chronic wounds reduce protease activity, increase fibroblast activity, and increase oxygenation. Hydrogen peroxide is slowly released from honey placed on a wound through the interaction of wound exudates with the honey's inherent glucose oxidase. This hydrogen peroxide is in sufficient concentration to be antibacterial, yet dilute enough to be non-toxic while promoting fibroblast proliferation and angiogenesis. Manuka honey also possesses non-peroxide antibacterial activity in what is called the Unique Manuka Factor (UMF) due to the presence of methylglyoxal.

Honey has been shown to contain a number of phenolic compounds, which are known to scavenge and remove reactive oxygen species (ROS) released by neutrophils. Honey suppressed oedema and leukocyte infiltration in a mouse model of neutrophilic inflammation. Monocytes cultured in the presence of honey produced a number of pro- and anti-inflammatory cytokines (e.g., tumor necrosis factor alpha (TNF-α), interleukin-1 beta (IL-1β), and interleukin-6 (IL-6)), which and may indicate modulation towards resolution in non-healing wounds.

Platelet-rich plasma (PRP) therapy has been gaining momentum as a bedside regenerative medicine procedure and has been used to stimulate regeneration of osteochondral defects, tendon/ligament injuries, and chronic dermal wounds (diabetic and pressure ulcers) in clinical studies. PRP is a simple and cost-effective method for collecting and concentrating platelets for the purpose of activating and releasing their growth factor-rich alpha and dense granules. Platelet dense granules release a number of growth factors and cytokines, including: platelet-derived growth factor (PDGF), transforming growth factor beta (TGF-β), vascular endothelial growth factor (VEGF), fibroblast growth factor (FGF), epidermal growth factor (EGF) and others. These growth factors and cytokines, in conjunction with the numerous factors contained in PRP, are known to accelerate cell migration and proliferation, promote ECM production, as well as play a role in macrophage phenotype and inflammation resolution. It has been previously demonstrated that the combination of Manuka honey and PRP/PRGF results in an acid activation of an array of growth factors; moving these growth factors from a latent form to a physiologically active form that is more readily utilized by cells and thereby increasing growth factor potency (Sell et al., International Journal of Biomaterials 2012 Article ID 313781).

Engineered tissue provides a promising alternative for replacement of defective tissue in which a live, natural tissue/support composition is generated from a construct made from a subject's own cells in combination with a support composition. A number of individual growth factors and/or cytokines have been used in an array of sustained release tissue engineering and regenerative medicine applications with positive results. The importance of keeping preparations rich in growth factors at wound sites and their sustained release has also been demonstrated. For example, tissue regeneration systems containing biomolecules for sustained biomolecule release include gas foaming porous plastic scaffolds, gelatin microspheres, plastic microspheres, alginate beads, and multilayered hydrogels. These systems can be difficult to process into structures for tissue ingrowth while also provide adequate biological activity and/or sustained release of the biomolecule.

Accordingly, there is a need for alternative tissue engineered structures that provide enhanced bioactivity and sustained biomolecule release for wound healing and tissue engineering.

### BRIEF DESCRIPTION

In one aspect, the present disclosure is directed to a tissue engineered scaffold including a fiber support and honey.

In one aspect, the present disclosure is directed to a tissue engineered scaffold including an electrospun fiber support and honey.

In one aspect, the present disclosure is directed to a method of preparing a tissue engineered scaffold comprising an electrospun fiber support and honey, the method comprising: preparing a solution comprising honey and a solvent; adding a fiber material to the solution; delivering the solution to an electrode; applying a voltage to the electrode; pumping the solution through the electrode; and collecting the fiber.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 shows scanning electron micrographs of PCL electrospun from solutions containing increasing amounts of honey or water (as a control) as described in Example 2.

FIG. 2 is a graph illustrating mean fiber diameters of electrospun scaffolds as measured from scanning electron micrographs as discussed in Example 2.

FIG. 3 is a graph illustrating the tensile modulus over time for scaffolds prepared using 20% honey, 20% water and pure HFP as discussed in Example 3.

FIG. 4 is a graph illustrating the simultaneous evaluation of cellular proliferation and chemotaxis as discussed in Example 4.

FIG. 5 is a graph illustrating bacterial clearance distance from scaffolds prepared using honey as discussed in Example 5.

FIG. 6 is a graph depicting the clotting time for scaffolds containing varying concentrations of PRGF and discussed in Example 7.

FIG. 7 is a graph depicting macrophage proliferation on scaffolds containing varying concentrations of honey as discussed in Example 8.

FIG. 8 is a graph depicting is a graph depicting fibroblast proliferation on scaffolds prepared using 10% honey and containing varying concentrations of PRGF as discussed in Example 9.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

In accordance with the present disclosure, tissue engineered scaffolds having a fiber support and honey and methods for preparing the tissue engineered scaffolds are described. The tissue engineered scaffolds can further include at least one biomolecule. The tissue engineered scaffolds can be used to promote cellular chemotaxis, enhance cell proliferation, enhance extracellular matrix production, increase angiogenesis, and provide antimicrobial activity. The nature of the tissue engineered scaffolds provides a template for cellular infiltration and can guide tissue regeneration. The tissue engineered scaffolds can be used in the treatment of dermal wounds (burns, chronic wounds, etc.) or as a tissue engineering scaffold in a wide range of applications.

### Tissue Engineered Scaffolds

In one aspect, the present disclosure is directed to tissue engineered scaffolds. The tissue engineered scaffolds include a fiber support and honey. The tissue engineering scaffolds can be prepared using methods that allow for the evaporation of the solvent. A particularly suitable method is electrospinning Other particularly suitable methods can be, for example, electroblowing, extrusion, sheets, and films.

Materials used to prepare the fiber support can be any fiber support materials known in the art. Suitable fiber support materials can be biodegradable (also referred to herein as “bioresorbable”) material, non-biodegradable material and combinations thereof As used herein, “bioresorbable” and “biodegradable” are used interchangeably herein to refer to a material that is biocompatible as well as degradable and/or absorbable by a subject. Biodegradable material is intended to be broken down (usually gradually) by the body of an animal, e.g. a mammal A bioresorbable material is intended to be absorbed or resorbed by the body of a subject, such that it eventually becomes essentially non-detectable at the site of application.

Fiber support material can be a synthetic polymer, a natural protein and combinations thereof Suitable synthetic polymers can be, for example, polycaprolactone (PCL), polydioxanone (PDO), poly (glycolic acid) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol), polymer nanoclay nanocomposites; a halogenated polymer solution containing metal compounds (e.g., graphite); poly(ethylenimine), grafted cellulosics, poly(ethyleneoxide), and poly vinylpyrrolidone; polystyrene (PS) and combinations thereof.

Suitable natural proteins can be, for example, silk fibroin, collagen, elastin, hyaluronic acid, gelatin, fibrinogen, chitin, chitosan, fibronectin and combinations thereof.

Fibers can include varying diameters depending on the type of material used to prepare the fiber support, the fabrication protocols, and the like. Fibers can have a diameter of about 5 μm or less. In another aspect, the fibers can have a diameter of about 0.5 μm to about 4.5 μm. Fiber diameter can be determined, for example, by scanning electron microscopy.

Any suitable honey can be used. Particularly suitable honey can be, for example, Manuka honey, medical-grade Medihoney, which is a Manuka honey product, and combinations thereof. Manuka honey is a specifically active Leptospermum honey from New Zealand. Suitable honeys can further include those having a high osmolarity and a high sugar content. Suitable honeys can further include those having an acidic pH. Particularly suitable acidic pH honeys are those having a low pH such as, for example, a pH of about 3.5 to about 4.5.

In another aspect, the tissue engineered scaffolds include a fiber support and honey, as described herein, and further include at least one biomolecule.

Suitable biomolecules can be, for example, growth factors, cytokines, bioactive lipids, immunoglobulins, and combinations thereof. Particularly suitable biomolecules can be, for example, platelet derived growth factor (PDGF), transforming growth factor beta (TGFβ), vascular endothelial growth factor (VEGF), fibroblast growth factor (FGF), epidermal growth factor (EGF), human growth factor (HGF), bone morphogenetic proteins (BMPs), insulin-like growth factors (e.g., IGF-1 and IGF-2), keratinocyte growth factor, connective tissue growth factor, chemotactic proteins, sphingosine 1-phosphate (S1P), various macrophage and monocyte mediators such as RANTES (Regulated upon Activation, Normal T-cell Expressed, and Secreted), tumor necrosis factor α (TNF α), interferon gamma (IFNγ), and granulocyte-macrophage colony stimulating factor (GM-CSF), lipoxin and combinations thereof. Suitable cytokines can be, for example, interleukins (e.g., IL-1-IL-36) and interferons (e.g., interferon type I, interferon type II, interferon type III).

The biomolecule can also be a preparation rich in growth factors (PRGF). PRGF can be prepared from blood or platelet rich plasma (PRP). To prepare PRGF, blood can be used to create PRP using methods known to those skilled in the art. For example, the HARVEST® SMARTPREP® 2 kit (Harvest Technologies Corp., Plymouth, MA) is a commercially available centrifugation system to create PRP. After obtaining PRP, the PRP is then subjected to a freeze-thaw-freeze (FTF) cycle for cell lysis. The FTF cycle can be performed by placing PRP in a −70° C. freezer, followed by thawing in a 37° C. water bath, and then returned to the −70° C. freezer. The frozen PRP is then lyophilized to create a dry PRGF powder that can be finely ground in a mortar and pestle prior to use.

In one aspect, the tissue engineered scaffold is porous. Suitable porosity includes from about 10% to about 95% porosity. Suitable pore sizes may be from about 10 μm to about 800 μm. Pore size may be determined, for example, by scanning electron microscopy. Porosity and pore size allow cells to migrate into and through pores and infiltrate into scaffold, allow culture medium circulation into and through pores, and allow exchange of nutrients and metabolic waste.

In another aspect, the tissue engineered scaffolds can include a plurality of cells. Cell types that can be used are, for example, endothelial cells, macrophages, adipose-derived stem cells, mesenchymal stem cells, embryonic stem cells, ligament fibroblasts, tendon fibroblasts, muscle fibroblasts, dermal fibroblasts, muscle cells and combinations thereof The cells can be autologous cells, allogeneic cells, or xenogeneic cells. “Autologous cells” refers to cells that are donated and received by the same subject. For example, cells are obtained from subject A, incorporated into the scaffold, and the cell-laden scaffold can be implanted into subject A. “Allogeneic cells” refers to cells that are donated by a subject that is different from the recipient subject; however, the donor subject and recipient subject are from the same species. For example, cells are obtained from subject A, incorporated into the scaffold, and the cell-laden scaffold is implanted into subject B. “Xenogeneic cells” refers to cells that are obtained from or donated by a species that is different than the recipient. For example, cells are obtained from species A, incorporated into the scaffold, and the cell-laden scaffold is implanted into species B.

In another aspect, the tissue engineered scaffolds can further be coated with a cell adhesion molecule. The cell adhesion molecule coating the tissue engineered scaffolds contacts the electrospun fibers making up the scaffold. The cell adhesion molecule can be, for example, fibronectin, vitronectin, collagen, RGD (arginine-glycine-aspartic acid) peptide, LDV (leucine-aspartic acid-valine) peptide, laminin and combinations thereof. Unique physical characteristics of electrospun fibers enhance adsorption of cell adhesion molecules, induce favorable cell to extracellular matrix interactions, promote in vivo-like three-dimensional adhesion and activate cell signaling pathways, maintain cell phenotype, and support cell differentiation.

In another aspect, the tissue engineered scaffolds can further be coated with other molecules such as, for example, recombinant and chemically synthesized proteins and peptides and nucleic acids (DNA and RNA).

### Methods of Preparing Tissue Engineered Scaffolds

In another aspect, the present disclosure is directed to a method of preparing a tissue engineered scaffold, wherein the tissue engineered scaffold has a fiber support and honey. The method includes preparing a solution having honey and a solvent; adding a fiber support material to the solution; delivering the solution to an electrode; applying a voltage to the electrode; pumping the solution through the electrode; and collecting the fiber.

The tissue engineered scaffold can be prepared using suitable fiber support materials and honeys as described herein.

The solution is prepared by dispersing honey in a solvent. The honey can be dispersed in the solvent using methods known by those skilled in the art such as, for example, sonicating, vortexing, mixing, stirring and combinations thereof A particularly suitable method is by sonication. The ratio of honey to solvent can be, for example, about 1% (volume/volume) to about 30% (volume/volume). The ratio of honey to solvent can also be, for example, about 1% (volume/volume) to about 20% (volume/volume). The ratio of honey to solvent can also be, for example, about 10% (volume/volume) to about 20% (volume/volume).

Any suitable solvent known by those skilled in the art may be used that is capable of dissolving the fiber support materials and providing a conducting fluid capable of being electrospun. Suitable solvents can be, for example, N,N-dimethyl formamide (DMF), tetrohydrofuran (THF), N—N-dimethyl acetamide (DMAc), chloroform, methylene chloride, dioxane, ethanol, 1,1,1,3,3,3 hexafluoroisopropanol (HFP) and combinations thereof A particularly suitable solvent is 1,1,1,3,3,3 hexafluoroisopropanol (HFP).

Upon dispersion of the honey in the solvent, a fiber support material is added to the solvent solution containing honey. The amount of fiber support material to be added can be from about 20 mg/mL to about 500 mg/mL, for example. One skilled in the art would readily understand that the amount of fiber support material to be added can depend on the type of support material and its particular characteristics.

After dispersion of the fiber support material in the solvent solution, the method includes delivering the solution to an electrode. Delivering the solution to an electrode can be by any method known to those skilled in the art. For electrospinning for example, the solution can be collected in a syringe that is then fitted with a blunt tip needle and a syringe pump, and delivered to an electrode. The solution can be pumped at a controlled flow rate ranging from about 2 mL/hour to about 4 mL/hour. A positive high voltage of from about +10 kV to about +30 kV can be applied to the electrode. The distance from the tip of the electrode to the collection substrate (such as a mandrel or a collecting plate) can be from about 5 cm to about 30 cm. A particularly suitable distance is about 15 cm.

In another aspect, the present disclosure is directed to a method of preparing a tissue engineered scaffold, wherein the tissue engineered scaffold has a fiber support; honey; and at least one biomolecule. In one embodiment, the method includes preparing a solution having honey and a solvent; adding a fiber material to the solution; adding at least one biomolecule to the solution; delivering the solution to an electrode; applying a voltage to the electrode; pumping the solution through the electrode; and collecting the fiber. In another embodiment, the method includes preparing a first solution comprising honey and a solvent; adding a fiber material to the first solution; preparing a second solution comprising at least one biomolecule; delivering the first solution to an electrode; delivering the second solution to a second electrode; applying a voltage to the first electrode and the second electrode; pumping the first solution through the first electrode and the second solution through the second electrode; wherein an outlet tip of the first electrode is positioned in proximity to an outlet tip of the second electrode; and collecting the fiber from the first electrode and the second electrode.

The tissue engineered scaffold can be prepared using suitable fiber support materials, honey, biomolecules and solvents as described herein.

In embodiments including the preparation of a first solution and a second solution, the preparation of the first solution and the second solution allows for the preparation of tissue engineering scaffolds in which the first and the second solutions combine only at the tip of the electrode. The preparation of a first solution and a second solution and delivery to a first electrode and a second electrode allows for the preparation of two distinct fiber types, and thus, result in a tissue engineering scaffold including the two distinct fiber types. Additionally, the preparation of a first solution and a second solution can be used with an annular electrospinning setup to produce a coaxial electrospun fiber.

In the method, an outlet tip of the first electrode is positioned in proximity to an outlet tip of the second electrode. The angle at which the outlet tip of the first electrode is positioned in proximity to the outlet tip of the second electrode can readily be determined by those skilled in the art. A particularly suitable angle at which the outlet tip of the first electrode is positioned in proximity to the outlet tip of the second electrode is a 90° angle.

The disclosure will be more fully understood upon consideration of the following non-limiting Examples.

### EXAMPLES

### Example 1

In this Example, tissue engineered scaffolds containing honey and platelet rich plasma/powdered preparation rich in growth factors (PRP/PRGF) were prepared.

PRP and PRGF were created from human blood. Briefly, fresh human whole blood from 3 donors was purchased (Biological Specialty Corp.), pooled, and used in a HARVEST® SMARTPREP® 2 (Harvest Technologies Corp., Plymouth, MA) centrifugation system to create PRP according to the manufacturer's protocol. PRP was then subjected to a freeze-thaw-freeze (FTF) cycle in a −70° C. freezer for cell lysis (centrifuge tubes containing PRP were placed in a −70° C. freezer for 24 hours followed by a 37° C. water bath for 1 hour, and then returned to the −70° C. freezer for 24 hours). Frozen PRP was then lyophilized for 24 hours to create a dry PRGF powder which was finely ground in a mortar and pestle prior to use.

Manuka honey was mixed with 1,1,1,3,3,3 hexafluoroisopropanol (HFP) in increasing ratios (1-30%). Since the honey was immiscible in the HFP, the solutions were placed in a bath sonicator for 30 minutes followed by vortexing for 1 min. Following dispersion of the honey, 15% polycaprolactone (PCL) was added to the solutions to serve as the polymeric backbone of the scaffold structures. Solutions were allowed to dissolve overnight prior to electrospinning.

Completely dissolved solutions were electrospun onto a rotating mandrel through an 18 gauge blunt tip needle using the following parameters: 4 ml/hour flow rate, +27 kV charging voltage, 8″ distance between the needle tip and mandrel. Scaffolds containing a combination of honey and PRGF were created in a similar fashion. Once honey (in varying concentrations) was dispersed in the HFP, PRGF was added in varying concentrations (1-100 mg/ml) and also sonicated/vortexed. PCL was then added to the honey/PRGF solution and electrospun using identical parameters as above.

### Example 2

In this Example, tissue engineering scaffolds were prepared using increasing concentrations of honey.

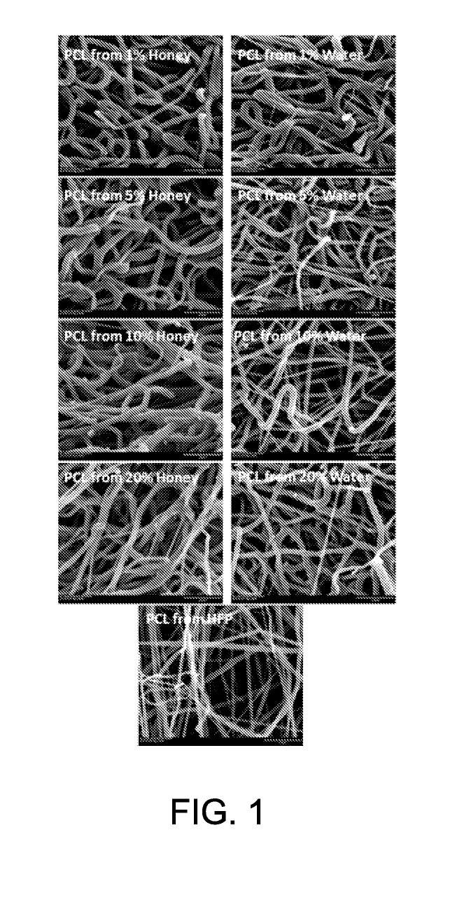

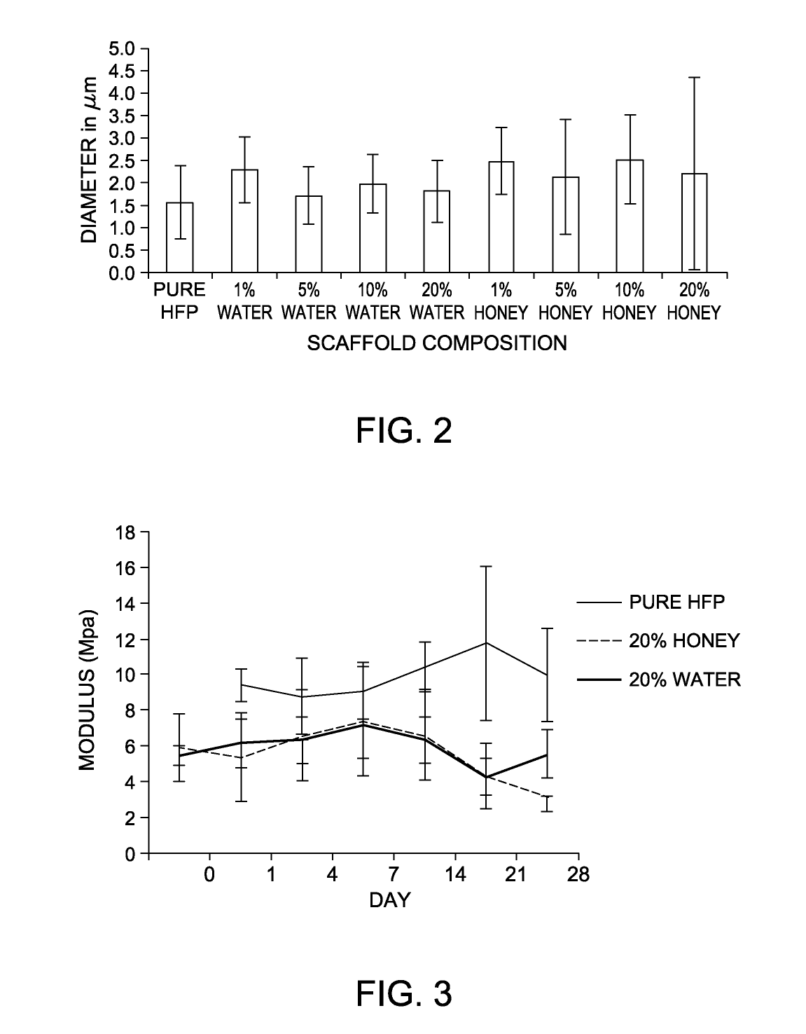

Specifically, Manuka honey was dispersed through sonication into hexafluoroisopropanol (HFP) in increasing concentrations (1-20% v/v), into which 150 mg/ml polycaprolactone (PCL) was dissolved. These solutions were then electrospun onto a rotating stainless steel mandrel to form a scaffold. Controls used increasing concentrations of water, sonicated and dispersed in HFP (1-20% v/v) with 150 mg/ml PCL dissolved, or 150 mg/ml PCL dissolved in pure HFP. Scaffolds were then imaged with scanning electron microscopy (SEM, FIG. 1, 1500× magnification, 20 micron scale bar). From these SEM images, fiber diameter and porosity of these scaffolds were measured using ImageJ image analysis program (FIG. 2).

Manuka honey was successfully dispersed in the HFP solution using sonication, where previous attempts (dissolving liquid honey, dissolving lyophilized honey, high speed vortexing, etc.) at dissolving honey in HFP had failed. This resulted in a viscous solution with a color similar to that of diluted Manuka honey. The solution was electrospun and created stable scaffolds that again contained a yellow honey-like appearance (more honey content resulted in a darker color). Image analysis of the SEMs demonstrated there to be no significant differences in fiber diameter with the inclusion of Manuka honey. Pore diameter demonstrated a similar result, although there was a non-significant trend of increased pore size with increased honey content.

### Example 3

In this Example, tissue engineered scaffolds were evaluated for mechanical testing and degradation.

Specifically, samples for mechanical testing and degradation evaluation were punched from electrospun scaffolds and placed in sterile phosphate buffered saline (PBS) under standard cell culture conditions. Samples were removed at specified intervals (day 0, 1, 4, 7, 14, 21, and 28) and uniaxially tested to failure on a Mechanical Testing Systems Criterion 42 testing system at an extension rate of 10 mm/min.

It was demonstrated that mechanical strength of the electrospun scaffolds decreased with increasing amounts of either water or honey. Surprisingly, it was demonstrated that the presence of the Manuka honey within the scaffolds did not significantly impact the rate of scaffold degradation over the 28 days of incubation (FIG. 3). PCL, while traditionally a slowly degrading polymer, breaks down through hydrolytic interactions which can be exacerbated through local decreases in pH. The presence of a large volume of Manuka honey (known to have a pH of 3.5-4.5) was expected to negatively impact the rate of scaffold degradation, and was therefore a surprising result that honey had no significant impact on the degradation rate. It was also extremely surprising that the high concentration honey containing scaffolds were completely stable out to the 28 day mark.

### Example 4

In this Example, cellular proliferation and chemotaxis on tissue engineered scaffolds were investigated.

Specifically, cellular proliferation and chemotaxis were evaluated simultaneously using a transwell assay and an MTS cell proliferation assay (CELLTITER 96® AQueous Non-Radioactive Cell Proliferation Assay, Promega Corp., Madison, Wisc.). 30,000 human dermal fibroblasts (hDF) were seeded in the insert of the transwell assay (polycarbonate membrane with 8 micron diameter pores) in 200 μL of complete media, while in the bottom of each test well was placed a 600 μL of complete media and a 10 mm diameter disk punched from each electrospun scaffold (control scaffold from pure HFP, a Manuka honey containing scaffold, a water additive scaffold), or a positive or negative control (platelet-rich plasma or Manuka honey supplemented media, respectively). Media in the insert was changed every other day to prevent nutrient depletion induced cellular chemotaxis, and both the insert and bottom well were then assayed using the MTS cell proliferation assay after 7 days of culture.

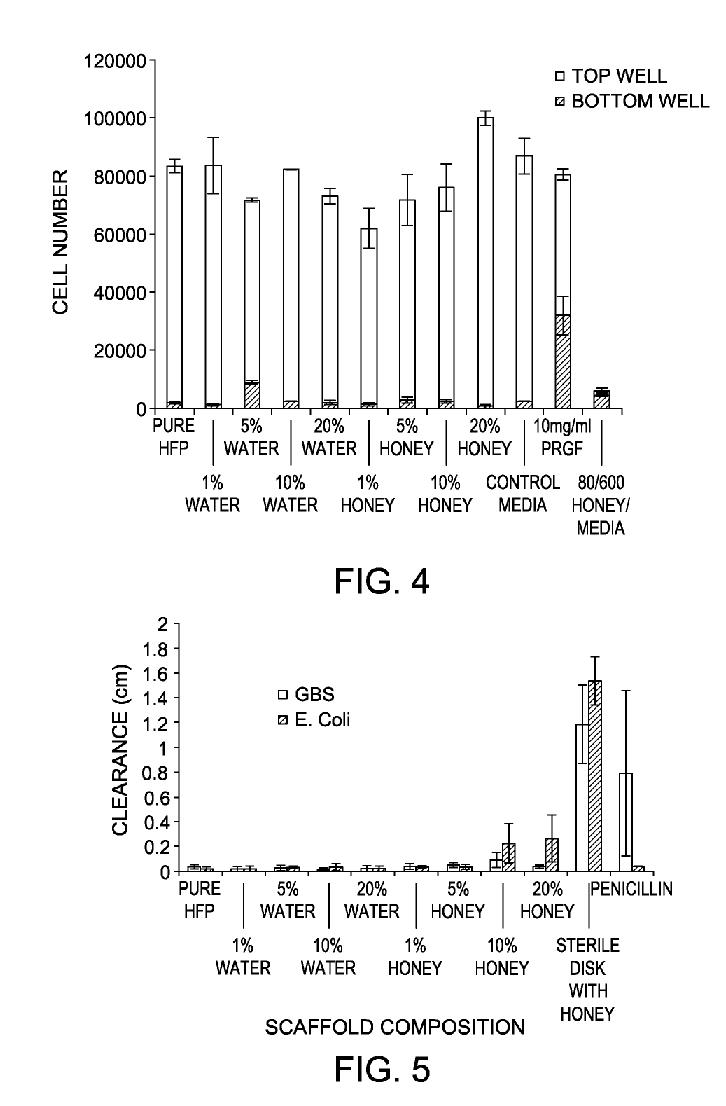

Similar to previously reported studies, the presence of Manuka honey was not effective in promoting cellular chemotaxis (i.e., the number of cells moving from the insert through the pores to the bottom well was minimal) compared to the platelet rich plasma supplemented positive control (FIG. 4). However, the presence of high concentration Manuka honey containing scaffolds (20% Manuka honey scaffolds) increased proliferation of hDF cells located within the insert compared to controls. Most impressive is the comparison of the 20% Manuka honey scaffold and the 80/600 honey/media negative control sample. This negative control was determined by calculating the volume of Manuka honey (80 μL) present in each 10 mm diameter disk of the 20% Manuka honey scaffold and then adding that amount of Manuka honey directly to the 600 μL of complete media. Therefore, there were equal amounts of Manuka honey present in each well of the 20% honey sample and the 80/600 honey/media sample; the difference being that in the 20% honey scaffold well the honey was released in a controlled fashion while in the 80/600 honey/media well all of the honey was present in a bolus fashion. The hDF numbers indicated that the bolus of Manuka honey, possibly due to the pH of the honey, had a cytotoxic effect while the sustained release fashion of the honey containing scaffold actually increased cellular proliferation (most likely due to a sustained release of sugars inherent to the honey). This data demonstrates the potentially cytotoxic impact of using a large amount of liquid Manuka honey in a wound site, compared to the sustained release potential afforded by the electrospun form of the Manuka honey.

### Example 5

In this Example, the effect of honey incorporated tissue engineered scaffolds on bacterial inhibition was determined

Specifically, the bacterial inhibition test was performed using Streptococcus agalactiae (Group B Streptococcus, GBS) and Escherichia coli to determine the effect of honey incorporated scaffolds on Gram-positive vs. Gram-negative bacteria, respectively. Agar gels were coated with either GBS (Gram-positive bacteria, similar to MRSA) or E. coli (Gram-negative bacteria, similar to Pseudomonas) and then 6 mm disks of scaffolds prepared using pure HFP, 1% (v/v) water, 5% (v/v) water, 10% (v/v) water, 20% (v/v) water, 1% (v/v) honey, 5% (v/v) honey, 10% (v/v) honey, and 20% (v/v) honey were placed on the bacteria seeded agar. Controls were a sterile 6 mm disk of filter paper coated with penicillin and a 6 mm disk coated directly with Manuka honey. The agar gels were incubated under standard conditions for 24 hours, after which time they were digitally imaged and the amount of bacterial clearance was determined using the ImageJ image analysis program.

Scaffolds containing high concentrations of Manuka honey (10% and 20%) demonstrated significant increases in clearing E. coli bacteria, although with less effectiveness against the Gram-positive GBS bacteria (FIG. 5). The Manuka honey control sample also demonstrated higher effectiveness against E. coli, while the penicillin control was highly ineffective against E. coli. These results demonstrated that even in the small volumes tested, the electrospun scaffolds containing the Manuka honey retained their antibacterial properties.

When coupled with the results from the hDF proliferation study, the electrospun scaffolds containing Manuka honey can serve as a viable scaffold for wound healing in that they are both antibacterial yet promote proliferation in human cell types, while the Manuka honey by itself, with undoubtedly more potent antibacterial properties, proves to be toxic to both bacteria and human cells alike. The amount of bacterial inhibition was directly related to the volume of Manuka honey present. Without being bound by theory, the antibacterial behavior of the Manuka honey scaffolds may be due to the presence of methylglyoxal inherent to Manuka honey. As the Manuka honey breaks down, it releases hydrogen peroxide, and will increase its antibacterial capabilities.

### Example 6

In this Example, varying amounts of preparation rich in growth factors (PRGF) were incorporated in honey+PCL solutions and were electrospun analyzed to determine spinnability of the solutions.

Specifically, solutions of 15% PCL, 10% honey, and increasing PRGF concentrations (0.1%, 0.5%, 1%, 2% and 3%) were formulated and electrospun. At 3% PRGF, the solution formed globules on the needle tip and formed fibers only intermittently, creating a scaffold that was more brittle, thinner, and more macroporous than scaffolds prepared using solutions with less than 3% PRGF. Solutions above 3% PRGF were not spinnable.

### Example 7

In this Example, honey-PCL scaffolds prepared with PRGF were analyzed for induction of blood clotting.

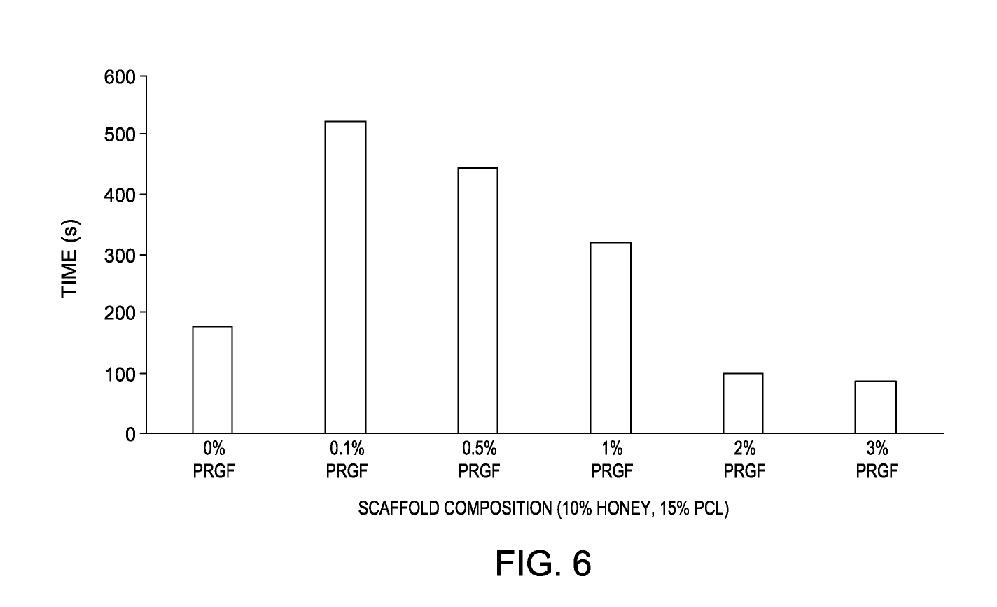

Specifically, 10% honey-15% PCL scaffolds with varying concentrations of PRGF (0%, 0.1%, 0.5%, 1%, 2% and 3%) were placed in bovine's blood in the presence of thrombin and CaCl₂. As depicted in FIG. 6, increasing PRGF concentration resulted in a decrease in clotting time. The only data point that does not fit this trend is the 0% PRGF scaffold, on which clotting was observed more quickly than on the 0.1%, 0.5% and 1% PRGF scaffolds.

### Example 8

In this Example, macrophage proliferation on honey-PCL scaffolds was analyzed.

Specifically, scaffolds were prepared using pure HFP, 1% water, 5% water, 10% water, 20% water, 1% honey, 5% honey, 10% honey and 20% honey. Macrophages were seeded at a cell count of 50,000 cells per well and cultured for 4 days. Cell counts were made at Day 1, Day 2 and Day 4.

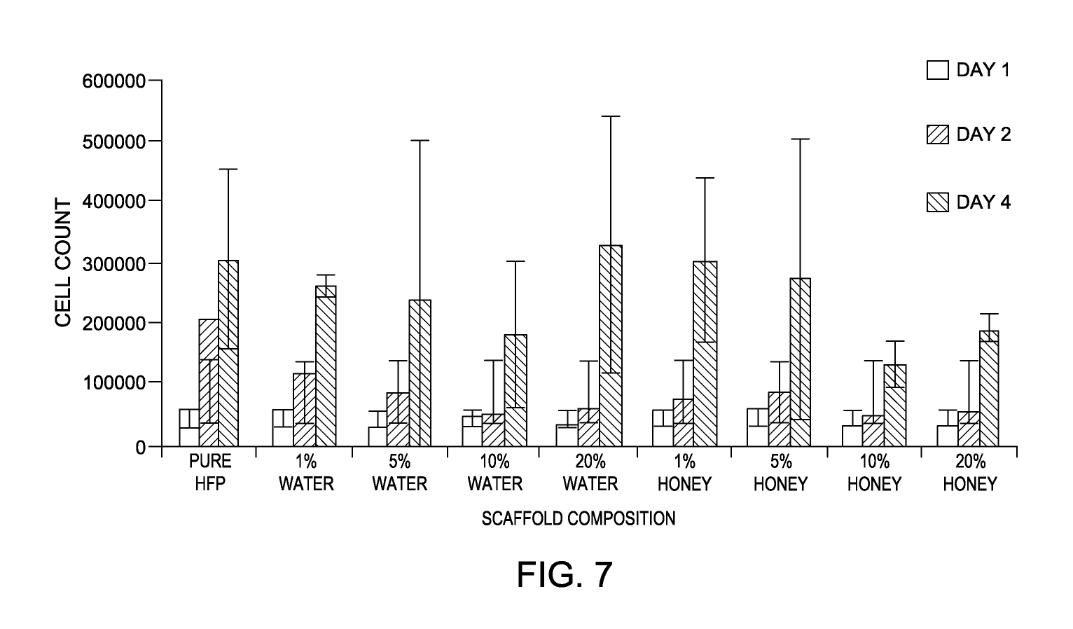

As depicted in FIG. 7, the scaffolds did not induce macrophage proliferation. Although macrophage number increased over the four day timeframe, the cell number remained relatively consistent between the water control scaffolds and the honey scaffolds. This result demonstrates that macrophages did not have a severe inflammatory response to the scaffolds. The lack of any substantial increase in macrophage proliferation indicates that the scaffolds are not overtly immunogenic.

### Example 9

In this Example, fibroblast proliferation on honey-PCL scaffolds was analyzed.

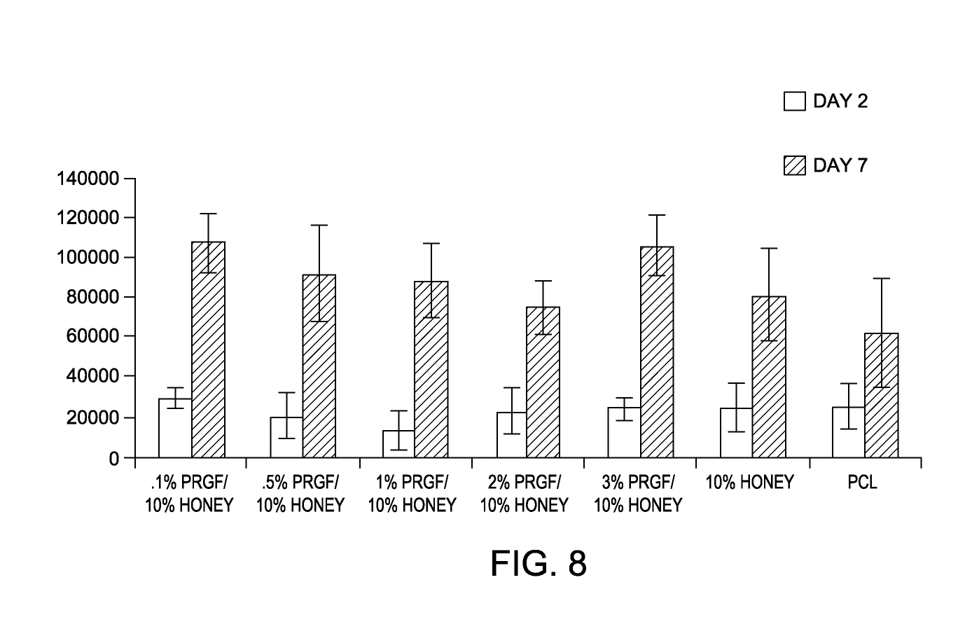

Specifically, 10% honey-15% PCL scaffolds with varying concentrations of PRGF (0.1%, 0.5%, 1%, 2% and 3%), 10% honey-15% PCL scaffolds and PCL-only scaffolds were prepared and seeded with 50,000 fibroblast cells per well and cultured for 7 days. Cell counts were taken at Day 2 and Day 7. As depicted in FIG. 8, an increase in cell proliferation at certain concentrations of PRGF relative to the control of 10% honey scaffold and the PCL-only scaffold control with no additives. These results demonstrated that PRGF content positively influenced fibroblast proliferation within and around the scaffold, promoting wound healing.

These Examples demonstrated the feasibility of an electrospun PDO/PCL/Manuka honey/PRGF scaffold as a dermal graft. In vitro testing showed an increase in fibroblast proliferation with both honey and PRGF incorporation, demonstrating the accelerated wound response resulting from the elution of these factors into the wound bed. Macrophage testing showed no severe immune reaction to this scaffold, and degradation testing enabled tuning the polymer ratio for proper scaffold degradation over the wound healing timeframe. Clotting tests demonstrated the retained clotting potential of the scaffolds, demonstrating the ability of honey scaffolds to improve hemostasis.

In view of the above, it will be seen that the several advantages of the disclosure are achieved and other advantageous results attained. As various changes could be made in the above devices and methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A tissue engineered scaffold comprising a fiber support and honey.

2. The tissue engineered scaffold of claim 1 further comprising at least one biomolecule.

3. The tissue engineered scaffold of claim 2, wherein the at least one biomolecule is selected from the group consisting of a growth factor, a cytokine, a bioactive lipid, an immunoglobulin, and combinations thereof.

4. The tissue engineered scaffold of claim 3, wherein the at least one biomolecule is a preparation rich in growth factors.

5. The tissue engineered scaffold of claim 1, wherein the fiber support is selected from the group consisting of an electrospun fiber support, an electroblown fiber support, an extruded fiber support, a fiber sheet, and a film support.

6. The tissue engineered scaffold of claim 1, wherein the fiber support comprises a material selected from the group consisting of a synthetic polymer, a natural protein and combinations thereof.

7. The tissue engineered scaffold of claim 6, wherein the synthetic polymers is selected from the group consisting of polycaprolactone (PCL), polydioxanone (PDO), poly (glycolic acid) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol), a polymer nanoclay nanocomposite; a halogenated polymer solution containing metal compounds (e.g., graphite); poly(ethylenimine), grafted cellulosics, poly(ethyleneoxide), and poly vinylpyrrolidone; polystyrene (PS) and combinations thereof.

8. The tissue engineered scaffold of claim 6, wherein the natural protein is selected from the group consisting of silk fibroin, collagen, elastin, hyaluronic acid, gelatin, fibrinogen, chitin, chitosan, fibronectin and combinations thereof

9. The tissue engineered scaffold of claim 1, wherein the honey is Manuka honey.

10. The tissue engineered scaffold of claim 1, further comprising a cell adhesion molecule.

11. The tissue engineered scaffold of claim 10, wherein the cell adhesion molecule is selected from the group consisting of fibronectin, vitronectin, collagen, an RGD (arginine-glycine-aspartic acid) peptide, a LDV (leucine-aspartic acid-valine) peptide, laminin and combinations thereof.

12. A tissue engineered scaffold comprising an electrospun fiber support and honey.

13. The tissue engineered scaffold of claim 12, wherein the honey comprises Manuka honey.

14. The tissue engineered scaffold of claim 12 further comprising at least one biomolecule.

15. The tissue engineered scaffold of claim 14, wherein the at least one biomolecule is selected from the group consisting of a growth factor, a cytokine, a bioactive lipid, an immunoglobulin, and combinations thereof.

16. The tissue engineered scaffold of claim 14, wherein the at least one biomolecule is a preparation rich in growth factors.

17. A method of preparing a tissue engineered scaffold comprising an electrospun fiber support and honey, the method comprising
preparing a solution comprising honey and a solvent;
adding a fiber material to the solution;
delivering the solution to an electrode;
applying a voltage to the electrode;
pumping the solution through the electrode; and
collecting the fiber.

18. The method of claim 17, further comprising adding at least one biomolecule to the solution.

19. The method of claim 17, wherein the honey is Manuka honey.

20. The method of claim 17, wherein the fiber material is selected from the group consisting of a synthetic polymer, a natural protein and combinations thereof
